(19) United States
(12) Patent Application Publication
(10) Pub. No.: US 2013/0267796 A1
(43) Pub. Date: Oct. 10, 2013

(54) SYSTEM AND METHOD FOR THE SIMULTANEOUS, NON-INVASIVE ESTIMATION OF BLOOD GLUCOSE, GLUCOCORTICOID LEVEL AND BLOOD PRESSURE

(71) Applicant: Enric Enric Monte Moreno, Barcelona (ES)

(72) Inventor: Enric Enric Monte Moreno, Barcelona (ES)

(73) Assignee: UNIVERSITAT POLITECNICA DE CATALUNYA, Barcelona (ES)

(21) Appl. No.: 13/991,034

(22) PCT Filed: Nov. 30, 2011
(86) PCT No.: PCT/ES2011/070831

§ 371 Date: May 31, 2013

(30) Foreign Application Priority Data
Dec. 1, 2010 (ES) P201031780

(51) Int. Cl. A61B 5/00

(52) U.S. Cl. CPC A61B 5/7235
USPC 600/301

(57) ABSTRACT

System and method for the simultaneous and non-invasive estimation of blood glucose, glucocorticoid and pressure levels. It comprises an activity module detector for a digitalized signal acquired by a sensor, representing the distal heartbeat of a person, selecting a segment of consecutive signal samples of fixed duration S_{window}(t) and using the same to generate sub-windows S_{frame}(t, n) of shorter duration; a signal processing module which receives the two signals S_{window}(t) and S_{frame}(t, n) and which delivers a vector X_{F} at its output with the parameters of a physiological model and module based on automatic learning, which receives the X_{F} vector and information on a person's characteristics and provides an estimation of blood glucose levels (BGL), systolic pressure levels (SPL), diastolic pressure levels (DPL) and glucocorticoid levels (GCL) at its output.

### FIELD OF THE INVENTION

The present invention initially refers to a system for the simultaneous and non-invasive estimation of blood pressure levels, glucocorticoid levels and glucose in the blood of a person, based on the waveform of the distal heartbeat.

A second aspect described in this invention is a method for the simultaneous and non-invasive estimation of glucose and glucocorticoid levels in the blood and the blood pressure of a person.

The estimation of glucose levels in blood is important in the control of diabetes mellitus since it requires daily assessment in order to avoid long-term complications.

The need for a non-invasive method for estimating glucose levels is justified by the size of the population affected and by the limitations of the systems based on carrying out the estimation by drawing blood. According to Wild et al (S. Wild et al, Global Prevalence of Diabetes, Diabetes Care 27 (5) (2004) 1047-1053), at least 171 million people around the world suffer from diabetes. Controlling glucose levels requires glucometers, disposable needles and test strips to be used. This control method is expensive and inconvenient. The regular control of glucose levels in blood can help to prevent complications associated with diabetes such as retinal, kidney and cardiovascular diseases.

Furthermore, given that this variable is measured in a routine way in intensive care units, a non-invasive measurement reduces the risk of infections for these people.

Moreover, according to K. Wolf-Maier et al (Hypertension Prevalence and Blood Pressure Levels in 6 European Countries, Canada and the United States JAMA 289 (18) (2003)), the prevalence of hypertension (defined as diastolic pressure above 90 mmHg and systolic pressure above 140 mmHg) was 28% of the North American population and 44% in Europe. Given that hypertension increases the risk of heart attack, heart failure, embolisms and kidney diseases, a non-mechanical, easy to use system for the estimation of blood pressure would improve the daily control of blood pressure levels.

Underlying glucose and blood pressure level are glucocorticoids, which indirectly control glucose and blood pressure levels. Glucocorticoids are a class of steroid hormones, which act on homeostasis levels of inflammatory processes and stress. References to this aspect of the effect of glucocorticoids can be found, for example, in Sapolsky, Robert; Lewis C. Krey and Bruce S. McEwen (25 Sep. 2000), “The Neuroendocrinology of Stress and Aging: The Glucocorticoid Cascade Hypothesis”. Science of Aging Knowledge Environment 38: 21 y en Sapoilsky, Robert; L. Michael Romero and Allan U. Munck (2000). “How Do Glucocorticoids Influence Stress Responses? Integrating Permissive, Suppressive. Stimulatory, and Preparative Actions”. Endocrine Reviews 21 (1): 55-89. The level of glucocorticoids acts on the autonomic nervous system and the effect on blood pressure and glucose levels is reflected in the form of distal heartbeat.

In addition to simultaneously estimating glucose and glucocorticoid levels in blood, as well as, systolic and diastolic pressure levels are justified by the population, which is at the same time affected by hypertension and diabetes. According to the “American Diabetes Association” (http://www.diabetes.org/diabetes-basics/diabetes-statistics), in 2004, 75% of adult diabetics had blood pressure levels over 130/80 mmHg or were taking medication for hypertension. Given that hypertension and the manifestation of diabetes is related to a person's lifestyle, especially to the levels of stress they must endure, the additional estimation of glucocorticoid levels will make it possible to determine the risks and treatment needs for this group of people. Another aspect which justifies the importance of determining glucocorticoid levels in the blood is that they are a good indicator of cardiac risk (Sher, L. Type D personality: the heart, stress, and cortisol, vol 98, May 2005, QJM: An International Journal of Medicine) and Gillmisal Gilder, et al. Complementary and Incremental Mortality Risk Prediction by Cortisol and Aldosterone in Chronic Heart Failure, Circulation 115: 1754-1761). Finally, a high glucocorticoid level is known to be related to the appearance of diabetes (M. McMahon, et al., Effects of glucocorticoids on carbohydrate metabolism, Diabetes/Metabolism Reviews Volume 4, Issue 1, pages 17-30, February 1988).

In this invention, distal heartbeat is used to simultaneously estimate glucose levels and glucocorticoid levels in blood, as well as, blood pressure. Given that the waveform of distal heartbeat reflects a person's physiological state, the distal heartbeat will be taken from the parameters describing this physiological state. Moreover, the physiological state of a person determines blood pressure level, glucocorticoid level and glucose levels in blood, where there is a significant interaction between these three variables. Therefore, glucocorticoid levels in the blood determine the state of the autonomic nervous system, which at the same time determines the heartbeat form, cardiac variability, distal blood supply, glucose and pressure levels. In order to estimate glucose, glucocorticoid and pressure levels, the use of a “machine learning” system is proposed, which may take advantage of the fact that the three magnitudes interact with one another to give a more precise estimation of the three values.

### BACKGROUND OF THE INVENTION

The physiological values, which justify the relationship between the waveform of distal heartbeat and the three variables to be estimated, will firstly be listed. Furthermore, alongside each physiological value, the type of signal treatment is described which facilitates the creation of a model containing this physiological information and that is incorporated in the system and method described in this patent.

a—Blood Viscosity and Blood Vessel Compliance:

The effect of viscosity and variation on blood vessel compliance is reflected in the extent to which the waveform of the distal heartbeat is subdued. This information may be obtained from the waveform of the distal heartbeat through spectral analysis and a model based on the true cepstrum of the waveform. The cepstrum is a magnitude, which facilitates the deconvolution and separation of the system excitation from the impulse response of the same (Childers, D. G. et al., “The cepstrum: A guide to processing,” Proc. IEEE, October 1977). Coefficients of the cepstrum are therefore used, which have been calculated based on the distal heartbeat and which characterize the heartbeat waveform, in order to separate the excitation component from the component corresponding to the capillary transfer function, as well as to blood viscosity. Another advantage of using the cepstrum is that the Euclidian distance for comparing various signals is well defined within the cepstral domain (Gray, A., et al. “Distance measures for speech processing,” IEEE Trans. on Acoustics, Speech and Signal Processing, October 1976). Another index from which information on the harmonic risk of the signal is derived is spectral entropy (P. Renevey, A. Drygajlo, Entropy based voice activity detection in very noisy conditions, in: EUROSPEECH-2001).

b—The Baroreceptor Reflex:

The baroreceptor reflex is a negative feedback system, which controls short-term changes in blood pressure. The baroreceptor reflex is evident in heart rhythm and the waveform of the distal heartbeat. It specifically modifies the spectrum of frequencies of the interval between heartbeats and the heart rate frequency variability, which indicates the state of the baroreceptor reflex (R. W. deBoer, et al. Hemodynamic fluctuations and baroreflex sensitivity in humans: a beat-to-beat model, Am J Physiol Heart Circ Physiol 253 (3) (1987) H680-689).

Furthermore, we know that the baroreceptor reflex is controlled in part by glucocorticoid levels, therefore the variables characterizing this reflex also provide information on glucocorticoid level (Quinkler M, Stewart P M. Hypertension and the cortisol-cortisone shuttle. J Clin Endocrinol Metab. 2003 June; 88(6):2384-92.). Given that the barometric reflex is governed by a non-linear equation and indirectly intervenes with the regulation of glucose, the functional model's estimation must be capable of inferring a non-linear function. This justifies the use of automatic learning techniques of either the “radial basis function” type, CART. “support vector machine” or improvements made via a group of approximating functions, as in the case of AdaBoost or of the bagging of classifiers. It also justifies the use of spectral characteristics of the cardiac variability and its energy profile in the characterization of the physiological state, which controls glucose, blood pressure and glucocorticoid levels.

c—Metabolic Syndrome:

Metabolic syndrome (M.-A. Cornier, et al., The Metabolic Syndrome, Endocr Rev 29 (7) (2008)) consists of hypertension, obesity and insulin resistance. There is likewise a feedback type interaction between metabolic system and neuroendocrine stress, which is manifested in increased cortisol levels and disturbances in the spectral components of cardiac variability (E. J. Brunner, et al. Adrenocortical, Autonomic, and Inflammatory Causes of the Metabolic Syndrome: Nested Case-Control Study, Circulation, November 2002; 106: 2659-2665). We also know that metabolic syndrome is reflected in cardiac variability (D Liao, et al. Multiple metabolic syndrome is associated with lower heart rate variability. The Atherosclerosis Risk in Communities Study Diabetes Care, December 1998; 21: 2116-2122) which justifies that, in addition to the description of the distal heartbeat waveform and the statistics on cardiac variability, variables which make it possible to characterize metabolic syndrome are used, namely age, weight and body mass index.

d—The Relationship Between Diabetes and Variability in Heart Rate Frequency:

We know that diabetes alters the variation in heart rate frequency (M. Ducher, et al., Noninvasive exploration of cardiac autonomic neuropathy. Four reliable methods for diabetes?, Diabetes Care 22 (3) (1999)). A relationship between glucocorticoid level and heart rate frequency alterations have also been identified in the case of diabetes (J. i. Davies, et al. Spironolactone impairs endothelial function and heart rate variability in patients with Type 2 diabetes. Diabetologia, Volume 47, Number 10, 1687-1694). Given that, there is a functional relationship between alterations in glucose levels due to diabetes and variability in heart rate frequency, the characteristic that will make it possible to describe this physiological relationship will be the power spectrum on the distance between heartbeats and the general statistics on heart rate frequency and on its variability. The power spectrum will be represented by the cepstrum.

e—Emotional States:

Emotional states such as anger, sadness, happiness, surprise, stress etc. alter values on blood pressure, glucose and glucocorticoid levels. The various emotional states are related to the characteristics on the power spectrum on heart rate frequency variability (R. McCraty, et al., The effects of emotions on short-term power spectrum analysis of heart rate variability, The American Journal of Cardiology 76 (14) (1995)). Furthermore, mood alterations, particularly in the case of depression, are related to anomalous levels of glucocorticoids and of changes in cardiac variations (Robert M. Carney, et al. Depression, Heart Rate Variability, and Acute Myocardial Infarction, Circulation. October 2001; 2024-2028). This physiological characteristic justifies the use of the power spectrum on the distance between heartbeats and a model, which captures the frequency components of the distance between heartbeats, as well as the use of general statistics on heart rate frequency.

f—Respiration and the Autonomic Nervous System:

Various studies have demonstrated that respiratory rate may regulate blood pressure and indirectly, blood glucose levels via the state of the autonomic nervous system (E. Grossman, et al., Breathing-control lowers blood pressure. J Hum Hypertens 15 (5) (2001)). In diabetic patients, glucose and blood pressure levels are closely linked (M. Schein, et al., High blood pressure reduction in diabetics with interactive device-guided paced breathing: Final results of a randomized controlled study, Journal of Hypertension 25 (2) (2007)). Furthermore, the presence of various types of glucocorticoids in the blood is known to act as stimulants in the respiratory system and in the control of the respiratory reflex (Tarja Saaresranta, et al. Hormones and Breathing, Chest December 2002 122:2165-2 182).

The index used to model the relationship between respiratory rhythm and the autonomic nervous system will be the rate of the power envelope of the distal heartbeat. Respiratory rate is known to be calculable based on the waveform of the distal heartbeat, for example with the signal taken by means of a pulse oximeter (P. Leonard, et al., Standard pulse oximeters can be used to monitor respiratory rate, Emerg Med J 20 (6) (2003)). This justifies using low frequency components of the energy measured in the short-term of the distal heartbeat waveform, in order to estimate the respiratory rate and its spectral components (P. Leonard, et al. A fully automated algorithm for the determination of respiratory rate from the photoplethysmogram. The Journal of Clinical Monitoring and Computing 20 (February 2006) 33-36(4)).

The preferred embodiment to obtain the distal heartbeat in this invention will be based on the signal from a photoplethysmogram. Given that said equipment is based on measuring the differential absorption of light in a tissue, several examples on the background for non-invasive estimation of glucose based on this measurement are given below.

To this effect, the following documents may be cited: U.S. Pat. No. 5,222,496, U.S. Pat. No. 5,515,847, US 2007/0213607, US 2005/0107676, US 2007/0123759, U.S. 60/16,435, US 2008/0111971, US 1995/5433197, US 2008/4014321.

An alternative to using light to measure glucose concentration is taking advantage of magnetic fields, as in US patent application 2009/7635331 based on the principle of magnetic resonance. Nevertheless, the operating principle is similar to previous cases in that it is based on estimating glucose by means of the differential absorption of light.

None of the previous patents use the distal heartbeat waveform obtained from the differential absorption of light, in order to estimate the glucose levels in blood or blood pressure levels.

A different approximation based on physiological principles appears in US 2009/027070700 A1, which is based on using a light absorption sensor, which detects the HO concentration in a person's breath. This physiological principle differs from those used to justify the present invention, in that it is based on measuring the concentration of a chemical component rather than on the distal heartbeat waveform.

Another kind of non-invasive measurement is based on measuring glucose by diffusion through the skin and sweat as in patent application US 2006/0004271 A1.

Another measurement based on physiological principles is that presented in patent application US 2005/6949070, which is based on the temperature difference between various points of the ear and the concentration of glucose in the blood. Given that the temperature gradient, depending on the concentration variation of glucose, is very small, 0.024 C per mg/dl, the results provided by this system have to be recalibrated over time and for different people.

In terms of background for measuring glucocorticoids, the methods known are based on blood analysis, urine analysis and saliva analysis. The present patent differs in that it does not require bodily fluids to be extracted in order to estimate glucocorticoid levels in the blood.

Another way to estimate the glucocorticoid concentration is by using reactants, as is the case in patent application US 2008/0118397 A1, which requires the reactive test strips to be changed, thus impeding a continued estimation and also requires saliva samples for each estimation. U.S. Pat. No. 6,833,274 B2 is also based on using reactants to measure cortisol levels in saliva, which presents the same limitations as the previous patent.

As far as the non-invasive estimation of cortisol is concerned, cortisol being a type of glucocorticoid, document US 2002/0019055 is known to describe a piece of equipment which measures the concentration of cortisol by using a reactant which is placed on the skin and reacts with the components present in the plasma.

One patent application based on fluorescent effects, US 2006/0105397 A1, presents the same limitations as the patents named above, since it requires bodily fluids, whether saliva, urine or plasma, to be extracted.

Patent ES 2336997 discloses the non-invasive measurement of blood pressure and ES 2338624 relates to the non-invasive measurement of glucose levels in blood. Although these two patents aim at a similar objective to that of this invention, they offer very different solutions. These two patents, ES2336997 and ES 2338624 explain that a non-invasive measurement of blood pressure and blood glucose is carried out (but not of glucocorticoid levels) and they differ from the present invention in the following ways:
The alleged measurement of blood pressure and glucose levels in blood is carried out separately and they do not take advantage of the interactions between the two magnitudes without taking into account that pressure and glucose depend on the same physiological parameters and that the values evolve over time dependently. Furthermore, the estimation system they propose, based on “random forests” (an algorithm based on a set of classifiers), does not make it possible to estimate simultaneously and take advantage of the interactions between, the two magnitudes since they are based on CART type decision trees (Leo Breiman, et al. Classification and Regression Trees. Wadsworth 1984). In addition, the prediction given by terminal nodes of the trees consists of one single scalar.
The activity of the digitalized signal emitted by a sensor (such as a photoplethysmogram) is not detected and the duration of the signal received is not controlled. This lack affects the system in two ways:
   The use of variable length signal windows increases the number of nodes on each “random forest” tree, therefore reducing performance in terms of the missing score estimation (MSE).
   Detecting the signal presence, guarantees that the estimation can be carried out using the signal coming from the sensor (SNR) with an acceptable level of quality. In addition, detecting the signal presence, as the present invention proposes, by means of an activity detector (signal presence in the communication route) it is also important, since it is well known that the measurements made using a photoplethysmogram are altered owing to the movements of the person on whom the measurement is being made. When the person on whom the measurement is being made moves, the sensor momentarily loses contact, which causes spurious peaks to appear and signal losses, which alter the estimated parameters and give rise to erroneous estimations.
The use of variable length windows in the two cited patents ES2336997 and ES 2338624 make the estimators of the ARMA (Autoregressive moving average model) model incorporated in the same to have an uncontrolled variability, owing to the fact that the ARMA parameters associated with similar waveforms, measured on different time scales, will not be the same, which increases the complexity of the decision trees associated with the system based on “random forests”, owing to the fact that each tree would need a greater number of nodes in order to be able to model a certain type of waveform.
In the measuring system of the cited patents ES2336997 and ES 2338624, the signal measured is modeled using a stochastic ARMA system. The use of these parameters as a classifier input is known to be erroneous, since the adequate metric is based on the prediction error rather than on the parameters. A detailed explanation of why it is erroneous to use ARMA parameters, particularly the AR embodiment preferred in the two patents ES2336997 and ES 2338624, as direct classifiers can be found in (Gray, A., et al. “Distance measures for speech processing,” IEEE Trans. on Acoustics, Speech and Signal Processing, October 1976) and in Itakura (Itakura, F., “Minimum prediction residual principle applied to speech recognition,” IEEE Trans. on Acoustics, Speech and Signal Processing, February 1975).
In the claims of cited patents ES2336997 and ES 2338624, the use of information on cardiac coherence is mentioned without being justified, whilst explicit references to the calculation of statistics on distance variation between heartbeats do not appear in the description and neither do references to a person's rate of breathing.
In patents ES2336997 and ES 2338624, Teager energy is calculated (J. F. Kaiser. Some useful properties of Teager's energy operators, Proc. ICASSP93, 1993) in order to model the physiological state of the person using AR parameters, without specifying which physiological aspect they wish to record.
In FIG. 4 of the two patents ES2336997 and ES 2338624, zero-cross calculation is mentioned as a way of entering the characteristics vector. However, neither its use nor the way of calculating it, are justified. It is important to highlight that in the two patents mentioned, zero-crosses are used as input for the “random forest”, which estimates blood pressure level in one patent and glucose level in the other patent.
Neither of the two patents ES2336997 and ES 2338624 use information on the internal structure of the signal observation window. In the present invention, this information is obtained from the sequence of frames and it is very important to model spectral energy variability correctly and be able to characterize the physiological state of the person, via the temporal evolution of the magnitudes of interest over a fixed period of time, which would be one minute in the preferred embodiment.

The present invention differs from the two patents cited, namely ES2336997 and ES 2338624, in the fact that in an estimation process or method, to model the photoplethysmographic signal it uses information from the “cepstrum”, a set of parameters in which the Euclidian metric is well defined and equivalent to the integral quadratic error of the logarithmic difference of the Fourier transform modules of the signals. The reason why this property is important is that in “machine learning systems”, taken into account in the present invention, the similarity measures are based on either Euclidian distance, in the case of “radial basis functions”, scalar products in the case of multilayer “perceptron” type neural networks and value comparison in the case of decision trees. The use of cepstral parameters is more adequate than the ARMA parameters, since:
a—In the case of “radial basis functions” the neurons in the hidden layer store either examples or averages of signals seen in training. Therefore, in each neuron, the Euclidian distance of the input with the example or average stored in the neuron is calculated and,
b—In the case of “multilayer perceptrons”, weights are proportionate to the input values seen during training (c.f. Tom M. Mitchell, Machine Learning, McGraw-Hill, 1997. Furthermore, the similarities are measured using a scalar product between the input and the weights.

The ARMA coefficients are a generalization of the AR coefficients and it does not make sense to calculate the distances of various coefficients in terms of comparing spectrums.

In the present invention, an estimation of the energy profile of the signal is used to estimate respiratory frequency. In the two patents, an AR model of Teager energy is used. Nevertheless, Teager energy does not react to low frequency components such as respiratory) frequency and furthermore, the correct way of modeling the low frequency component of the energy profile is not by comparing the AR parameters but rather by using a residual prediction error obtained by filtering the input signal with a whitening filter based on these parameters.

In the present invention, the energy profile of the signal is calculated using an estimator based on averages of the square of the signal (that is to say through a low pass filtering the energy) which provides the individual's respiratory component profile. This aspect of estimating the respiratory component is not considered in the two cited patents, ES2336997 and ES 2338624.

In the present invention, the zero crosses are used in the signal presence detection modules since the zero crosses, in the case of a distal heartbeat signal being present with a low amount of noise, will have a very limited value margin, whilst in the case of noise or signal absence, they would have high values. In the present invention, this information is not used as input for the “machine learning” based system.

### DESCRIPTION OF THE INVENTION

The present invention proposes a method for the simultaneous and non-invasive estimation of glucose in blood, glucocorticoid levels and blood pressure levels, based on a distal heartbeat waveform and acquired from a sensor (1), particularly a photoplethysmogram type sensor, which emits a digitalized signal, characterized in that it comprises the following stages:
Detection of said digititalised signal activity by selecting a segment of some consecutive samples from said digitalised signal, of a fixed duration, referred to as S_{window}(t) and the generation based on said segment of a sub-window or frame sequence, referred to as S_{frame}(t, n), shorter in duration than that of S_{window}(t), wherein the index i indicates the sample number within a frame and n is the frame number, making it possible to eliminate the useless segments of the signal comprising the initial transition, false clicks, signal losses, noise and saturation;
Processing the two signals S_{window}(t) and S_{frame}(t, n), containing the distal heartbeat waveform in a fixed duration segment and using these signals to generate a vector X_{F} containing the parameters of a physiological model; and
Feeding said X_{F} vector and information on a person's characteristics into a model (5) based on automatic learning which provides an output estimation of blood glucose levels (BGL), systolic pressure levels (SPL), diastolic pressure levels (DPL) and glucocorticoid levels.

In order to implement the abovementioned method, the invention proposes a system comprising the following three modules, as shown in FIG. 1.

The distal heartbeat waveform is obtained via a sensor (1). The digitalized signal will be a sequence referred to as S_{Distal Heartbeat}(t). This signal is the input for the module (2) for signal activity detection (AD), that is to say signal presence in the communication route. The AD module selects a fixed duration (t) segment of S_{Distal Heartbeat}(t). This signal segment is obtained by means of: a) a local signal presence and/or absence classifier, which also detects signal losses, which may occur as a result of the person moving and b) a finite state automaton, which filters false positives and false negatives. The aim of this module (2) is to guarantee the presence of a signal having sufficient quality in order to carry out the estimation whilst also ensuring that this signal is of fixed duration or, in other words, normalizes (in order to reduce variability in the prediction model estimation). In the preferred embodiment of the present invention, the signal will be obtained from a photoplethsmogram type sensor and the segment will be one minute in duration of clean signal.

This segment shall be referred to as S_{window}(t). S_{window}(t) is used to generate a sequence of sub-windows, referred to as S_{frame}=(t, n), which is of a shorter duration than that of S_{window}(t). In order to avoid confusion about the sub-windows, they shall be referred to as frames. In S_{frame}(t, n) the index i indicates the sample number within a frame and n represents the frame number. In the preferred embodiment of the invention, S_{window}(t) will be divided into 5-second frames with a 50% overlap. In this preferred embodiment, the number of frames in one window is N_{frame}=24.

The two signals S_{frame}(t, n) and S_{window}(t) are the input to the module (4) of signal processing (TS). This module (4) calculates the parameters, which describe the physiological state to which reference is made in the background section. The module (4) output of (TS), together with information on the person's (3) characteristics, including at least gender, age and body mass index, serve to generate an X_{F} vector which contains the parameters describing the physiological state as well as the person's (3) characteristics.

The X_{F} vector is the input to the module (5), which is a system based on “automatic learning” (Machine Learning) whose output is the estimation of blood glucose levels (BGL), systolic pressure levels (SPL), diastolic pressure levels (DPL) and glucocorticoid levels (GCL). In the preferred embodiment, the glucocorticoid type would be cortisol.

The system based on “automatic learning” must be capable of dealing with heterogeneous input and approximating a non-linear function linking the vector X_{F} with the variables of interest (BGL, SPL, DPI., GCL) based on examples. Another requirement is that it should be capable of taking advantage of the common information and the interactions between the three variables in order to estimate the same. A system combining the outputs of various regression methods will therefore be used, following the ideas of either:
a—‘AdaBoost’ (Freund Y., et al., A decision-theoretic generalization of on-line learning and an application to boosting. Journal of Computer and System Sciences. 55(1), 1997)
b—‘Bagging Predictors’ (Breiman L., Bagging Predictors. Machine Learning, 1996).

AdaBoost is an algorithm for training committees of regressors. The regressors may be of various kinds, whether decision trees, multilayer neural networks, “radial basis functions” or “Support Vector Machines”. The preferred embodiment would be a variant of “AdaBoost” formed by basic regressors of the “radial basis functions” type. The structure of this “automatic learning” block would therefore be a committee of regressors based on “radial basis functions”, each element of the committee being trained by means of an AdaBoost algorithm. This algorithm carries out the training of a series of regressors sequentially with the criteria that each additional estimator uses a biased version of the training base with regard to the base elements with which the previous classifiers had worse performance. One characteristic of “radial basis functions” is that they can take advantage of the fact that the magnitudes to be estimated correlate with one another in order to improve the estimation. As explained in the section on physiology, there is an interaction between blood glucose levels, diastolic and systolic pressure levels and the level of glucocorticoids in blood, which is reflected in the components forming the X_{F} vector. The neural networks, for example of the type of “radial basis functions”, are known to improve performance if they are trained to calculate various functions simultaneously among which a functional type relationship exists, as explained in (Machines That Learn from Hints. Y. S. Abu-Mostafa. Scientific American, 272(4):64-69, April 1995) and in (Reed, R. D. Neural Smithing: Supervised Learning in Feedforward Artificial Neural Networks (Bradford Book); MIT Press: 1999, pag. 275).

Below, the invention will be described in greater detail with the help of the attached drawings, which serve as a non-limiting example thereof.

### BRIEF DESCRIPTION OF THE DRAWINGS

FIG. 1a is a general block diagram of the system of this invention, which implements the method for the simultaneous, non-invasive measurement of glucose and glucocorticoid levels in blood, as well as blood pressure.

FIG. 1b is a more detailed representation of the connection, inputs and outputs of the signal acquisition modules and signal activity (presence) detection modules of the system in this invention.

FIG. 2 shows a segment of signal, 5 seconds in length S_{Distal Heartbeat}(t) acquired by the sensor in FIG. 1.

FIG. 3 shows the block diagram of the AD activity detection module.

FIG. 4 is a diagram of the finite-state automaton used in the AD module.

FIG. 5 illustrates the transition norms between states of the finite-state automaton employed in the AD module.

FIG. 6 is a diagram representing the acquisition of the sequences used to calculate the collective parameters.

FIG. 7 is a diagram representing the acquisition of the overall parameters.

### DETAILED DESCRIPTION OF THE DRAWINGS

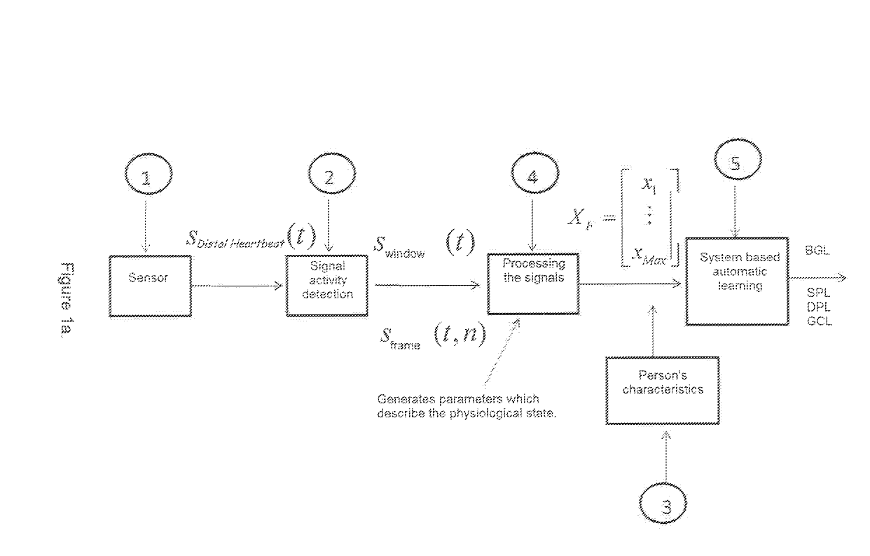

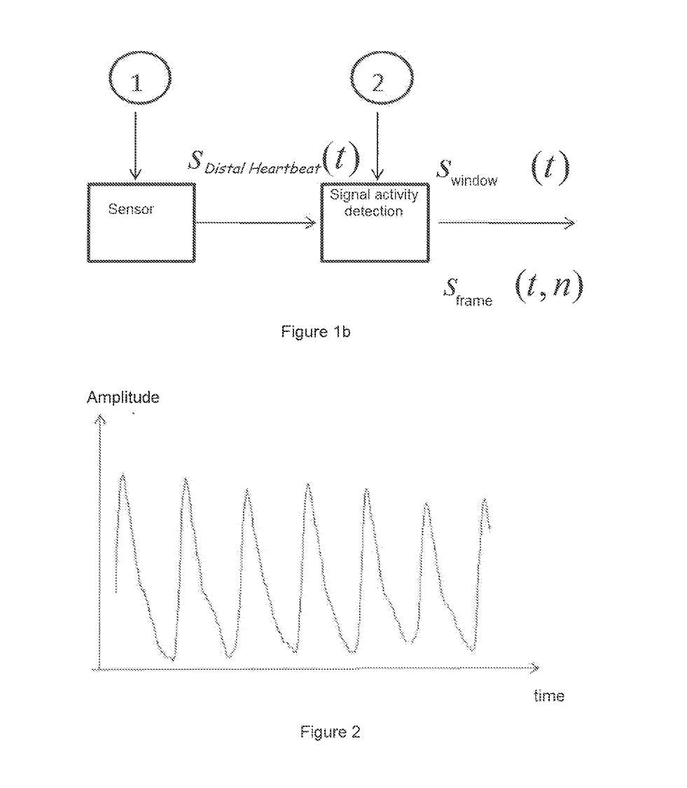

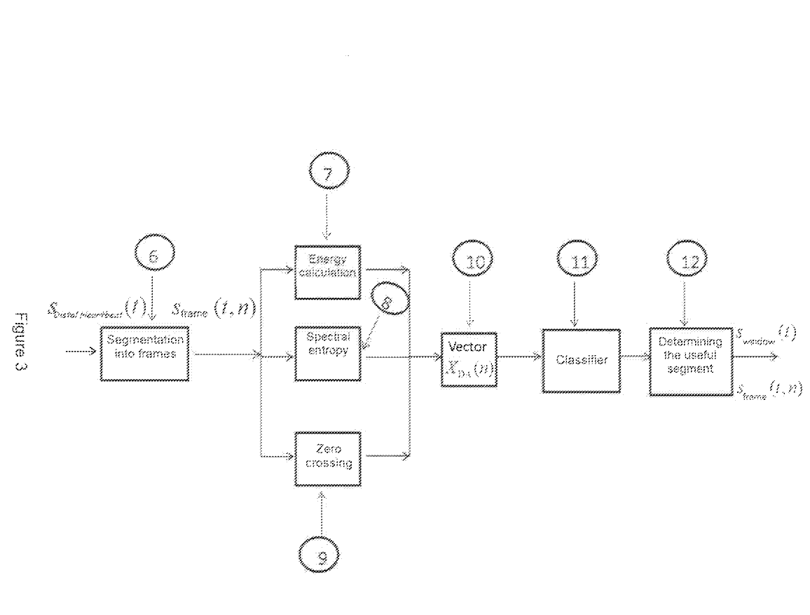

The invention consists of a system and method for carrying out an estimation of glucose levels in blood (BGL), systolic pressure levels (SPL), diastolic pressure levels (DPI.) and glucocorticoid levels (GCL). FIG. 1 shows a block diagram of the proposed system. The signal S_{Distal Heartbeat}(t) used to estimate the parameters of the physiological model is captured by a sensor (1), which, in the preferred embodiment, would be the plethysmographic type, which may be optical, acoustic or mechanic. The preferred embodiment of the invention will be carried out using a pulsioximetric system (SpO2). This type of sensor supplies a signal proportional to the absorption difference of reduced hemoglobin, relative to oxy-hemoglobin. This absorption difference generates a waveform proportional to the distal heartbeat. FIG. 2 presents an example of a 5-second signal segment. This digitalized signal is the input to a signal processing module (4) which, together with the information on the person's characteristics (3) is used as the input to a module (5) with a system based on “automatic learning”, whose output is the estimation of the value of the three variables, BGL, SPL, DPL and GCL. The systems (2, 4, 5) are implemented by a CPU formed by micro-controllers, DSP, FPGA or software run on a general use computer or mobile telephone/PDA or earphone.

Below is a detailed explanation of each of the modules referred to.
Signal activity detection module (2).

The aim of the AD module is to eliminate those signal segments, which are not useful, such as: the initial transition, false clicks, signal losses, noise and saturation etc. The result is a segment of consecutive signal samples of sufficient quality and normalized duration, in order to carry out the estimation. The output of this module will consist of the signals S_{window}(t), and S_{frame(t,} 1), S_{frame}(t, 2), . . . S_{frame}(t, N_{frame}) which will contain the distal heartbeat waveform in a fixed duration segment and its evolution by segments. The AD module (2) uses some parameters it has in common with the signal treatment module (4). The parameters used to determine whether the signal measured S_{Distal Heartbeat}(t) is useful (that is to say it corresponds to a distal heartbeat) are also used in the signal treatment module (4).

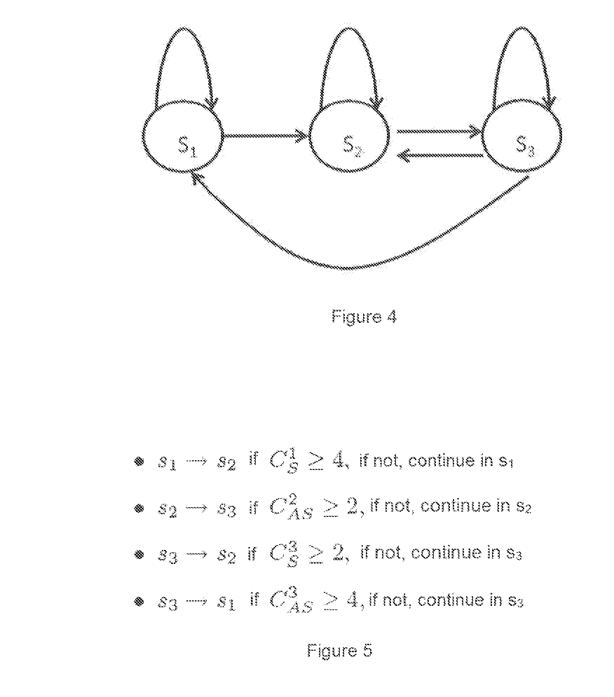

The AD module (2) comprises the following parts (see FIG. 3):
A sub-module (10), which calculates a vector of features X_{AD}(n) associated with each frame S_{frame}(t, n).
A decision function based on a classifier (11) which decides one of the two categories, “signal” or “absence of signal” for each frame.
A block (12), which determines the useful signal segment. This block has a finite-state Automaton (FSA), (FIG. 4) and a sub-module, which determines when the FSA has detected a number of consecutive frames corresponding to the desired duration of the useful signal segment. The output of this block are the signals S_{window}(t) and S_{frame}(t, 1), S_{frame}(t, 2), . . . , S_{frame}(t, N_{frame}).
   The AD module (2) uses the signal S_{Distal Heartbeat}(t) as input and continuously creates a set of frames S_{frame}(t, n) of lesser duration than that of S_{window}(t) which, in the preferred embodiment, would be 5 seconds long with a 2.5 second overlap between S_{frame}(t, n) and S_{frame}(t, n−1). The index I indicates the sample number within a frame and n refers to the frame number.

Below, the operations carried out to calculate the vector (FIG. 3) X_{AD}(n) are described.

### Energy Calculation (Sub-Module 7)

The energy of each frame makes it possible to determine whether the signal level corresponds to that of the useful signal. The output of this sub-module (7) will consist of three parameters:
E_{frame}^{μ}(n), corresponding to the average energy of the frame and calculated using the formula:

$${E}_{frame}^{p} ( n ) = \frac{1}{{L}_{frame}} {\sum }_{t = 1}^{{L}_{frame}} {( {S}_{frame} ( t , n ) - \frac{1}{{L}_{frame}} {\sum }_{t = 1}^{{L}_{frame}} {S}_{frame} ( t , n ) )}^{2} \qquad ( I )$$

Wherein L_{frame} is the total number of samples in the frame.
E_{frame}^{σ}(n)
E_{frame}^{σ}(n) corresponds to the standard deviation of the square of each sample and is calculated using the formula:

$${E}_{frame}^{a} ( n ) = \frac{1}{{L}_{frame}} {\sum }_{t = 1}^{{L}_{frame}} {( {{S}_{frame} ( t , n )}^{2} - {E}_{frame}^{a} ( n ) )}^{2} \qquad ( II )$$

E_{frame}^{σ}(n) corresponds to the square bias of each sample and is calculated using the formula:

$${E}_{frame}^{skew} ( n ) = \frac{\frac{1}{{L}_{frame}} {\sum }_{t = 1}^{{L}_{frame}} {( {{S}_{frame} ( t , n )}^{2} - {E}_{frame}^{u} ( n ) )}^{2}}{{( {E}_{frame}^{u} ( n ) )}^{3 \text{/} 2}} \qquad ( III )$$

### Spectral Entropy Calculation (Sub-Module 8).

The spectral entropy H_{frame}^{E}(n) is a function calculated for each frame which takes a high value for signals with flat spectrum of frequencies without spectral peaks, such as those which characterize low energy areas with transitory and/or clicks. Moreover, for harmonious tones and signals, this scalar adopts low values. Therefore, it is an adequate indicator of the useful signal presence because the distal heartbeat is characterized by having significant harmonic components. The properties of this indicator are explained in detail in P. Renevey, A. Drygajlo, Entropy based voice activity detection in very noisy conditions, in: EUROSPEECH-2001 and in J.-L. Shen, et al., Robust entropy-based endpoint detection for speech recognition in noisy environments, in: Proc. ICSLP98.

Spectral entropy is calculated in the following way:

Firstly, the Fast Fourier Transform (FFT) of the frame is calculated:

Xⁿ=FFT(s_{skew}(t,n),L_{FFT}) (IV)

Wherein L_{FFT} represents the length of the Fast Fourier Transform.
The energy spectrum of the frame is then normalized:

$${P}_{X}^{k} ( n ) = \frac{\vert {X}^{n} [ k ] {\vert}^{2}}{{Σ}^{{L}_{FFT}} \vert {X}^{n} [ j ] {\vert}^{2}} k = 1 , … {L}_{FFT} \qquad ( V )$$

Wherein the index k represents the k^{th} component of the Fast Fourier Transform of the frame.

$${H}_{frame}^{a} ( n ) = {\sum }_{k = 1}^{{L}_{FFT}} {p}_{n}^{k} ( n ) log ( {p}_{n}^{k} ( n ) ) \qquad ( VI )$$

### Zero Crosses (Sub-Module 9)

The use of this parameter is justified because, in the absence of a useful signal, the signal zero crosses a high number of times per second, whilst, in the presence of a heartbeat, the number of zero crosses would correspond to the heart rhythm and would be of approximately a zero cross per second. The preferred embodiment of the zero cross calculation Z_{frame}^{a}(n) will be carried out subtracting the average value S_{frame}(t,n) in order to eliminate the continuous component before counting the number of times the signal crosses a zero threshold.

Generating the X_{AD}(n) Vector (Sub-Module 10)

For each frame, the parameters calculated in sub-modules (7), (8) and (9) will be grouped together in a vector which will be the input of a classifier (11), which will decide whether or not the n^{th} frame corresponds to a useful frame:

X_{AD}(n)=[E_{frame}^{μ}(n),E_{frame}^{α}(n),E_{frame}^{skew}(n),H_{frame}^{s}(n),Z_{frame}^{c}(n)]^{T} (IV)

### Classifier (Sub-Module 11)

This sub-module (11) has a classifier whose output for each frame is an index, which indicates whether or not the frame pertains to one of the two categories: “useful signal” or “absence of signal”. This classifier is trained with a database previously labeled with the category to which each frame belongs. The type of classifier to be used may be of the k-nearest neighbor type, linear discriminants, quadratic discriminants, decision trees and support vector machines. The preferred embodiment will be a Fisher discriminant type classifier. The embodiment and training details on this kind of classifier are described in T. Hastie, et al., The Elements of Statistical Learning, Springer, 2001.

### Determining the Useful Segment (Sub-Module 12)

The input of this sub-module (12) will be the sequence of categories to which each frame belongs. This sequence is the input for a finite-state automaton (FIG. 4) and serves to determine segments of consecutive frames of useful signal. This machine has the following states:
s1=“absence of useful signal”
s2=“useful signal”
s3=“ambiguous area”
   Each state of the machine will have counters defined as follows:
C_{S}ⁱ: number of consecutive frames in the i^{th} state classified as “useful signal”
C_{AS}ⁱ: number of consecutive frames in the i^{th} state classified as “absence of signal”

FIG. 5 presents the norms, which determine the finite-state automaton making a transition from one state to the next.

The thresholds with which the counters are compared C_{S}ⁱ and C_{AS}ⁱ in order to decide on the transitions are determined based on examples. The criterion used to determine the thresholds is by minimizing the number of vectors S_{window}(t) with areas of non-useful signal in a training base. The preferred embodiment of this invention will use the thresholds presented in FIG. 5.

Those consecutive stretches found in states s2 and s3 will be considered useful signal. The output of the AD module consists of signals
S_{window}(t) and S_{frame}(t, 1), S_{frame}(t, 2), . . . , S_{frame}(t, N_{frame}). The output signals are entered when the number of consecutive frames in states s2/s3 is such that the total cumulated duration is equal to that prearranged for calculating the parameters for the physiological model. When signal loss is produced during the calculation or peaks of spurious signal appear, the counter starts over again so that the information it enters is made up of only useful signal. The preferred embodiment of the present invention uses durations of one minute for the segment covered by S_{window}(t). In the preferred embodiment, the frames will be 5 seconds long. For this reason, the duration covered by the set of frames and S_{frame}(t, 1), S_{frame}(t, 2), . . . , S_{frame}(t, N_{frame}) will also be 1 minute, with a 50% overlap between frames. The total number of frames will be Nframe=24. The number of frames is determined by the fact that the frames are sub-segments of the signal S_{window}(t).

### Signal Processing Module (4)

The signal processing module function serves to generate the X_{F} vector containing the parameters, which characterize the person's physiological state. The parameters formed by the X_{F} vector will be of two types:
Overall parameters calculated based on S_{window}(t)
Aggregated parameters of the characteristics calculated in the sequence S_{frame}(t, 1), S_{frame}(t, 2), . . . , S_{frame}(t, N_{frame}). The latter will serve to model the (dynamic) short-term evolution of several physiological parameters and the respiratory rhythm.

The preferred embodiment of spectral analysis applied in the present invention is justified below.

As mentioned in the background section, the physiological aspects controlling glucose and glucocorticoid levels in blood, as well as blood pressure levels become evident in the information on the spectral frequency contained in the cardiac signal. Therefore, some of the parameters of the physiological state model will consist of spectral type information. There are numerous techniques, which make it possible to carry out the spectral analysis of a sequence. Non-parametric periodogram type models are used and mentioned particularly in the bibliographic references given in the background of this invention. Although using a periodogram to represent the physiological information is viable, as is its use in this document, to calculate spectral entropy, the information contained in the spectral analysis will also be obtained via cepstral analysis. This choice is justified by the following reasons:
a) Cepstral analysis makes it possible to obtain information on the excitation of a system and its spectral response. The differences between systems and excitations may be calculated using the Euclidian distance between cepstral coefficients. The Euclidian distance between cepstral coefficients is equivalent to calculating the integral of the squared module of the logorhythmic difference in Fourier transforms. Furthermore, comparing the systems when they are characterized by AR or ARMA coefficients cannot be carried out using the Euclidian distance between coefficients because it is not equivalent to the difference between the squared module of Fourier transforms. This is why it was decided that the spectral information content be characterized indirectly using the cepstral coefficients. Underlying this argument is the fact that “machine learning” systems operate by comparing inputs with centroids using Euclidian distances or in the case of “multilayer perceptron” using a scalar product. It is known that a Euclidian distance between vectors can be decomposed as a sum of the square norm of each of the elements minus the scalar product. This is why representing the information using cepstral parameters is adequate.
b) When the impulse response of the system, which generates the signal, has a lesser duration than a periodic excitation period, the cepstral coefficients make it possible to characterize the frequency of the excitation. This justifies using them to characterize cardiac variability at the same time as distal vascularization.

The preferred embodiment in this invention will be the use of the cepstral coefficient vector, given that it contains the same information as the power spectrum and has fewer parameters, which facilitates the improved performance of the automatic learning system. The preferred method for calculating the cepstral coefficients of each sequence will be carried out using the recursive algorithm described in Nonlinear filtering of multiplied and convolved signals, Oppenheim, et al. Proceedings of the IEEE, 1968.

### Overall Parameters

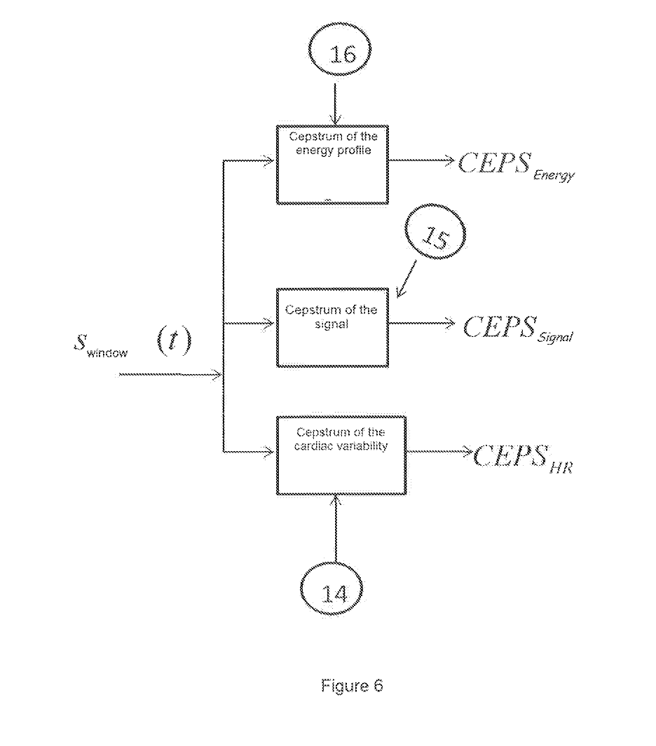

The overall parameters referred to are calculated in accordance with the proposal of this invention, based on S_{window}(t) and provide information about the spectrum of frequencies of three distal heartbeat variables (FIG. 6). In this invention, the preferred embodiment for estimating the spectrum of frequencies will be a parametric model based on cepstral coefficients.

In order to model the person's hemodynamic and distal vascularity state, the cepstral coefficients of S_{window}(t) (15) are calculated using the Oppenheim recursive algorithm. The result is a vector of coefficients referred to as CEPS_{signal}. The preferred order of this embodiment is of 7 coefficients.

In order to model cardiac variability (14) based on the sequence S_{window}(t), a new sequence is created that will consist in the instant period, understood as the distance (number of samples) between each distal heartbeat peak. This sequence shall be referred to as S_{Dist. Peaks}(t) and will coincide in duration with the number of beats in S_{window}(t). As shown in FIG. 2, it will be the distance in time between maximums. The preferred way of obtaining the sequence S_{Dist. Peaks}(t) consists in subtracting its average value from S_{window}(t) and calculating the distance between alternate zero crosses on the resulting sequence. The cepstral coefficients will be assigned to the vector CEPS_{HR}. The preferred order in this embodiment was 6 coefficients, obtained by means of the Oppenheim recursive algorithm.

In order to model respiratory frequency, the energy profile of S_{window}(t) (16) will be calculated. There are various possible methods for calculating this information, be it by means of wavelets (P. Leonard, et al., A fully automated algorithm for the determination of respiratory rate from the photoplethysmogram. The Journal of Clinical Monitoring and Computing 20 (February 2006)) or by means of the calculation based on low-pass filtering the squared high waveform. The latter would be the preferred method in this embodiment owing to the fact that the signal supplied by the AD has little noise contamination and no fluctuations produced by artifacts of the measurement and to the fact that there are fewer calculation requirements. The preferred method would be subtracting the average value from S_{window}(t) and finding the square value each one of its samples, before filtering the resulting sequence through a low pass filter. In the preferred embodiment, this filter would be of Chebychev type II, order 8 with a cutoff frequency of 1/20. The sequence resulting from the previous process is used to calculate the cepstral parameters, which are assigned to the CEPS_{Energy} vector. In this embodiment, the preferred order was of 6 coefficients, obtained by means of the Oppenheim recursive algorithm.

### Aggregated Parameters

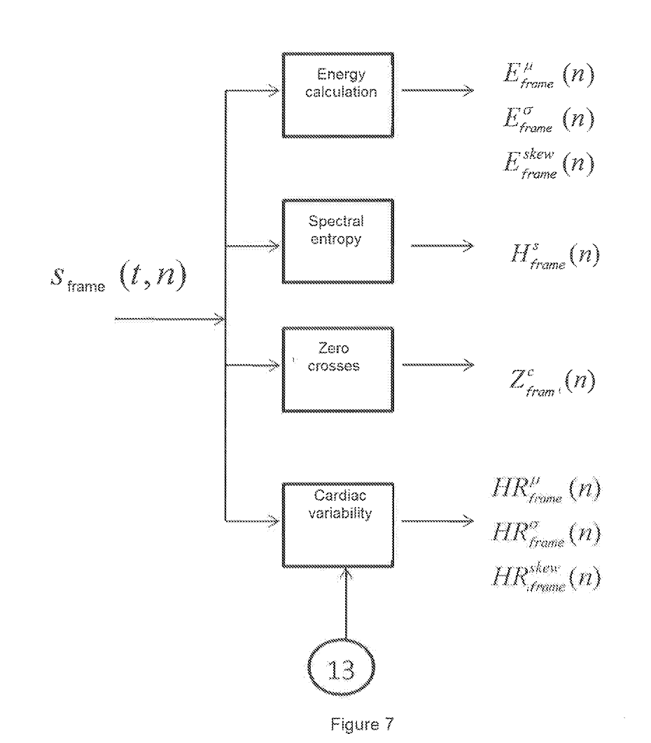

The aggregated parameters are calculated based on the sequence of consecutive frames S_{frame}(t, 1), S_{frame}(t, 2), . . . , S_{frame}(t, N_{frame}) and provides information on the evolution of the person's physiological state throughout the measurement window (FIG. 7). The calculation of the aggregated parameters uses information employed in the AD module. This is justified because these parameters, in addition to characterizing the person's physiological state, make it possible to determine whether a certain frame has a useful signal.

The first set of aggregated parameters is related to the energy of the frame. For each frame, the parameters E_{frame}^{B}(n), E_{frame}^{σ}(n), E_{frame}^{skew}(n) are calculated using formulas (I), (II) and (III). These parameters summarize the statistical characteristics of the energy in each frame. Based on these sequences, the following aggregated parameters are calculated:

$$\begin{matrix}{LogE}^{u} = \frac{1}{{L}_{frame}} {\sum }_{n = 1}^{{L}_{frame}} [ Log ( {E}_{frame}^{u} ] ( n ) ) & ( V ) \\ {LogE}^{a} = \frac{1}{{L}_{frame}} {\sum }_{n = 1}^{{L}_{frame}} [ Log ( {E}_{frame}^{a} ] ( n ) ) & ( VI ) \\ {E}^{akew} = \frac{1}{{L}_{frame}} {\sum }_{n = 1}^{{L}_{frame}} {E}_{frame}^{skew} ( n ) & ( VII ) \\ {CEPS}_{B}^{u} = CEPS ( {E}_{frame}^{u} ( 1 ) , … , {E}_{frame}^{u} ( {L}_{frame} ) , Order ) & ( VIII )\end{matrix}$$

Whereby Log E^{μ}, Log E^{σ}, E^{skew} are scalar, whilst CEPS_E^{μ} is an Order length vector calculated over the sequence of average values of the energy in each frame. In the preferred embodiment, the Order value will be 6.

The spectral entropy H_{frame}^{s}(n) will be calculated using formulas (IV), (V) and (VI). This parameter gives an indication of the signal's spectral purity. The average and the cepstral coefficients calculated based on the spectral entropy sequence of the frames, will be used as aggregated value.

$$\begin{matrix}{H}^{a} = \frac{1}{{L}_{frame}} {\sum }_{{L}_{frame}}^{{L}_{frame}} {H}_{frame}^{a} ( n ) & ( IX ) \\ {CEPS}_{u}^{s} = CEPS ( {H}_{frame}^{s} ( 1 ) , … , {H}_{frame}^{s} ( {L}_{frame} ) , Order ) & ( X )\end{matrix}$$

In the preferred embodiment, the Order value will be 6.

In terms of cardiac frequency, the intermediate values HR_{frame}^{μ}(n), HR_{frame}^{σ}(n), HR_{frame}^{skew}(n) will be calculated at frame level and are three sequences, which model the statistics of the instant distal heartbeat period. These values are calculated for all of the frames in the analysis segment, that is to say for n=1, . . . , N_{frame}. The calculation is carried out by creating an intermediate sequence S_{Dist peaks}^{(t,n)} from the signal, which will consist of the distance between distal heartbeat peaks. The three following sequences are firstly calculated:

$${HB}_{frame}^{u} ( n ) = \frac{1}{{L}_{DistPeaks}} {\sum }_{t = 1}^{{L}_{DistPeaks}} {S}_{DistPeaks} ( t , n ) \qquad ( XI )$$

Whereby L_{Dist. Peaks} is the number of samples of S_{Dist. Peaks}(t,n).

$$\begin{matrix}{HR}_{frame}^{a} ( n ) = \frac{1}{{L}_{DistPeaks}} {\sum }_{t = 1}^{{L}_{DistPeaks}} ( {S}_{DistPeaks} ) {( t , n )}^{2} - {{HR}_{frame}^{a} [ ( n ) )}^{2} & ( XII ) \\ {HR}_{frame}^{skew} ( n ) = \frac{\frac{1}{{L}_{frame}} {\sum }_{t = 1}^{{L}_{frame}} {( {{S}_{DistPeaks} ( t , n )}^{2} - {HR}_{frame}^{a} [ ( n ) ) ]}^{2}}{{( {HR}_{frame}^{a} ( [ n ) ) ]}^{\frac{3}{2}}} & ( XIII )\end{matrix}$$

Based on these sequences, the following added parameters are calculated:

$${HR}^{u} = \frac{1}{{L}_{frame}} {\sum }_{n = 1}^{{L}_{frame}} {HR}_{frame}^{a} ( n )$$

$$\begin{matrix}{HR}^{u} = \frac{1}{{L}_{frame}} {\sum }_{n = 2}^{{L}_{frame}} {HR}_{frame}^{u} ( n ) & ( V ) \\ {HR}^{a} = \frac{1}{{L}_{frame}} {\sum }_{n = 0}^{{L}_{frame}} {HR}_{frame}^{u} ( n ) & ( VI ) \\ {HR}^{skew} = \frac{1}{{L}_{frame}} {\sum }_{n = 2}^{{L}_{frame}} {HR}_{frame}^{skew} ( n ) & ( VII ) \\ {CEPS}_{{HR}^{u}} = CEPS ( {HR}_{frame}^{u} ( 1 ) , … , {HR}_{frame}^{u} ( {L}_{frame} ) , Order ) & ( VIII )\end{matrix}$$

In the preferred embodiment, the Order value will be 6.

These parameters characterize the spectral content of the cardiac frequency. Generating the X_{F} vector.

The X_{F} Vector (see FIG. 1a) is the output of the signal-processing module (4) and contains the set of parameters, which model a person's physiological state, as well as their physical characteristics such as gender, age and body mass index, etc. This vector will be the input of the module (5), which estimates the four output variables of the system by means of a system based on “automatic learning”.

$${X}_{AD} ( n ) = {[ \begin{matrix}{CEPS}_{signal} , {CEPS}_{HR} , {CEPS}_{Energy} , {LogE}^{v} , {LogE}^{a} , {E}^{skew} , \\ {CEPS\_E}^{u} , {H}^{s} , {CEPS\_H}^{s} , {HR}^{u} , {HR}^{a} , {HR}^{skew} , {CEPS\_HR}^{u} , \\ Age , Gender , Body mass index\end{matrix} ]}^{T} \qquad ( IV )$$

### Resistant Strength Relative to Sensor Change:

One significant aspect of the system, object of the present invention, consists in making the estimation independent from the sensor, in such a way that when one sensor is substituted by another, the estimation does not change. When substituting one sensor for another, even when using the same model, differences in the sensors' frequency response are generally produced, which may alter the measurements, thereby giving rise to erroneous estimations. For this reason, a cepstral subtraction process is carried out on the variables represented by cepstral coefficients. Cepstal subtraction is a common technique employed to offset the eftfects produced when changing microphones in speech recognition systems (L. R. Rabiner, B.-H. Juang, Fundamentals of Speech Recognition, Prentice Hall, 1993).

### “Automatic Learning” Module (5)

The “automatic learning” module uses the X_{F} vector as input and delivers as output the three variables of interest. It is a module, which implements a regression between the input X_{F} and the variables BGL, SPL, DPL, GDL. The algorithm used must be able to approximate a non-linear function, to provide ways of controlling the over-generalization effect and be able to learn the function even if the data contains noisy and/or inexact values. Furthermore, another requirement of this module is that the function obtained does not depend on the person and does not need to be recalibrated over time.

For this reason, the use of a system, which averages the outputs of various regression methods, is proposed, following ideas of an “AdaBoost” nature (Freund Y. et al., A decision-theoretic generalization of on-line learning and an application to boosting. Journal of Computer and System Sciences, 55(1), 1997). ‘Bagging of Classifiers’ (Breiman L. (1996): Bagging Predictors. Machine Learning, 24(3)), o ‘random forest’ (L. Breiman, Random forests, Machine Learning 45 (1) (2001)).

The preferred embodiment of the system based on automatic learning will be a “committee of predictors”, trained by means of the “AdaBoost” algorithm. The basic predictor in the “committee of predictors” type system will preferably be a “radial basis function” type neuronal network, which takes advantage of the interactions between pressure values, glucocorticoid levels and glucose levels in order to improve estimations. Given that each neuron in the hidden layer calculates a Euclidean distance of the input using a reference obtained during the training, the use of a cepstrum type parameterization is the most adequate for this type of estimator. As an alternative, “machine learning” systems such as “Support Vector Machines”, CART or multilayer “perception” systems may be used as predictors. Cepstrum type parameterization is also adequate, because these systems are based on either the use of distances or scalar products. The distal heartbeat form will preferably be measured using a plethysmograph.

A screen may be incorporated into the embodiment of the invention in order to visualize data, as well as a connection/keyboard to introduce the person's characteristics and control orders from the piece of equipment used. It has at least one acoustic, mechanical and/or optical probe, which provides the distal heartbeat signal, and the blocks (2, 3, 4, 5) are implemented in a system processor, either a CPU., micro-controller, DPS, FPGA, conventional computer, mobile telephone or PDA or earpiece.

The invention also proposes that pushbuttons or control panels are arranged in accordance with the state of the art, in order to activate and control the piece of equipment being used, as well as batteries and/or access to an external power source.

Likewise, the invention proposes the use of information transmission means, be it from the sensor or from the piece of equipment being used to carry out the estimation, to other systems, whether computers and/or medical diagnostic equipment via either serial port, USB, wireless connection or local network.

It goes without saying that within the present invention, as many alterations as desired may be made in the details or form, provided that they are comprised within the essence of the invention, as specified in the following claims.

1. System for the simultaneous and non-invasive estimation of glucose in blood, glucocorticoid levels and blood pressure, based on a person's distal heartbeat waveform, acquired from a sensor, which provides a digitalized signal, comprising:
an activity detection module for said digitalized signal, which selects a segment of consecutive samples from said digitalized signal, with a fixed duration and which are referred to as S_{window}(t) and, based on the same, generates a sequence of sub-windows or frames referred to as S_{frame}(t, n) which are shorter in duration than those of S_{window}(t), wherein the index t indicates the sample number within a frame and n represents the frame number;
a signal processing module which receives the two signals S_{window}(t) and S_{frame}(t, n) containing the waveform of the distal heartbeat in a fixed duration module, and which provides an X_{F} vector as output, which in turn contains the parameters of a physiological model; and
an automatic learning based module to which said X_{F} vector is fed alongside information on the characteristics of the person and which provides an estimation of blood glucose levels (BGL), systolic pressure levels (SPL), diastolic pressure levels (DPL) and glucocorticoid levels (GCL) as output.

2. System, according to claim 1, wherein said sensor is of the plethysmograph type selected from optical, acoustic or mechanical, including a pulse oximeter system (SpO2) and in that said activity detection module comprises:
a frame segmentation sub-module which provides said S_{frame}(t, n) sequence;
a sub-module to calculate the energy of each frame;
a sub-module to calculate the spectral entropy of each frame; and
a sub-module to detect zero crosses,
using said sub-modules to generate an X_{AD}(n) vector, which is fed to a classifier sub-module, which implements a decision function and whose index for each frame indicates whether the frame belongs to a “useful signal” category or corresponds to an “absence of signal”.

3. System according to claim 2, wherein said classifier sub-module is associated with a database labeled with the category to which each frame belongs for its training.

4. System, according to claim 3, wherein said classifier is selected from the group consisting of: k-nearest neighbor, linear discriminants, including the Fisher discriminant, quadratic discriminants, decision trees and “support vector machines”.

5. System, according to claim 2, wherein said activity detection module also includes a sub-module in its output for determining the useful segment the sequence of categories, to which each frame belongs, receives as input and said sub-module integrates a finite-state automaton used to determine segments of consecutive frames of useful signal, comprising the following states:
s1=“absence of useful signal”
s2=“useful signal”
s3=“ambiguous area”
and each state of the automaton have counters defined as follows:
C_{S}ⁱ: number of consecutive frames in the i^{th} state classified as “useful signal”; and
C_{AS}ⁱ: number of consecutive frames in the i^{th} state classified as “absence of signal”.

6. System, according to claim 1, wherein said automatic learning module uses a committee of predictors trained by means of the “AdaBoost” algorithm, using a “radial basic function” type neuronal network as a basic predictor of the committee of predictors, which takes advantage of the interactions between the pressure values, glucocorticoid level and glucose level in order to improve estimations.

7. Method for the simultaneous and non-invasive estimation of glucose in blood, glucocorticoid levels and blood pressure, based on a person's distal heartbeat waveform, acquired from a sensor, which provides a digitalized signal, comprising:
detecting the activity of said digitalized signal by selecting a segment of consecutive samples of said digitalized signal, of fixed duration and referred to as S_{window}(t) and using said segment to generate a sequence of sub-windows or frames referred to as S_{frame}(t, n) and of lesser duration than those of S_{window}(t), wherein the index t indicates the sample number within a frame and n represents the frame number making it possible to eliminate those signal segments which are not useful, comprising the initial transition, false clicks, signal losses, noise and saturation;
processing the two signals S_{window}(t) and S_{frame}(t, n), which contain the distal heartbeat waveform in a fixed duration segment and based on the same, generating an X_{F} vector, which contains the parameters of a physiological model; and
feeding said X_{F} vector and information on a person's characteristics into a module based on automatic learning and which provides an estimate of blood glucose levels (BGL), systolic pressure levels (SPL), diastolic pressure levels (DPL) and glucocorticoid levels (GCL) as output.

8. Method, according to claim 8, characterized in that claim 7, wherein said activity detection stage comprises:
segmenting the digitalized signal into frames S_{window}(t) providing a sequence of S_{frame}(t, n);
calculating the energy of each frame;
calculating the Fast Fourier Transform (FFT) of the frame for the spectral entropy of each frame before normalizing the energy spectrum of the frame and;
detecting zero crosses,
using these energy and spectral energy values to generate an X_{AD}(n) vector per frame, which is fed into a classifier sub-module, which in turn implements a decision function and whose index for each frame indicates whether the frame belongs to a “useful signal” category or corresponds to an “absence of signal”.

9. Method according to claim 8, characterized in that wherein said segmentation of S_{window}(t) is carried out in 5 second frames with a 50% digitalized signal overlap.

10. Method according to claim 8, wherein said zero cross detection is carried out by using the average value of S_{frame}(t, n) to eliminate the continuous component before counting the number of times the signal crosses the zero threshold.

11. Method according to claim 8, wherein for each frame, the parameters of energy, spectral entropy and zero crosses are grouped together in a vector, which will be the input of a classifier, which will decide whether or not the n^{th} frame corresponds to a useful frame:
X_{AD}(n)=[E_{frame}(n),E_{frame}^{σ}(n),E_{frame}^{skew}(n),H_{frame}^{s}(n),Z_{frame}^{c}(n)]^{T}

12. Method according to claim 11, wherein said classifier is selected from the group consisting of: k nearest neighbor, linear discriminants, including the Fisher discriminant, quadratic discriminants, decision trees and support vector machines and in that the classifier is trained with a database previously labeled with the category to which each frame belongs.

13. Method, according to claim 9, wherein in order to determine a useful segment, the sequence of categories to which each frame obtained at the output of said classifier sub-module belongs, is fed into a sub-module, which contains a finite-state automaton applied to certain segments of consecutive frames of useful signal, comprising the following states:
s1=“absence of useful signal”
s2=“useful signal”
s3=“ambiguous area”
and each state of the machine will contain counters, defined as follows:
C_{S}ⁱ: number of consecutive frames in the i^{th} state, classified as “useful signal” and;
C_{AS}ⁱ: number of consecutive frames in the i^{th} state classified as “absence of signal”.

14. Method, according to claim 8, wherein the signal processing stage entails generating an X_{F} vector containing the parameters, which characterize a person's physiological state, using overall parameters calculated based on S_{window}(t) and aggregated parameters of the characteristics calculated in the sequence S_{frame}(t, 1), S_{frame}(t, 2), . . . , S_{frame}(t, N_{frame}), which are useful for modeling the short-term evolution of various physiological parameters and respiratory rate, using the cepstral analysis to obtain information on the spectral content.

15. Method according to claim 14, wherein the average value of each sequence of cepstral parameters is subtracted to offset the effect of the specific sensor, calculating an average value of the cepstrums, each time a sensor is changed, for each group of parameters and storing said average value to carry out the subtraction during the signal processing stage.

16. Method, according to claim 7, wherein said module based on automatic learning receives the X_{F} vector at its input, along with the person's physical characteristics, including at least gender, age and body mass index and enters the three variables of interest at its output: blood glucose levels (BGL), glucocorticoid levels (GCL) and blood pressure levels (SPL and DPL) applying an algorithm, which implements a non-linear regression between said X_{F} input and the three variables cited, selecting a regression method, which models the interaction between the three variables.

17. Method, according to claim 16, wherein said automatic learning uses a committee of predictors trained by means of the “AdaBoost” algorithm, using a “radial basis function” type neuronal network as a basic predictor of the committee of predictors, which takes advantage of the interactions between pressure values, glucocorticoid levels and glucose level in order to improve estimations.
